(19) United States
(12) Patent Application Publication
(10) Pub. No.: US 2025/0140351 A1
(43) Pub. Date: May 1, 2025

(54) METHOD FOR STRUCTURAL ELUCIDATION OF SMALL MOLECULE COMPONENTS OF A COMPLEX MIXTURE, AND ASSOCIATED APPARATUS AND COMPUTER PROGRAM PRODUCT

(71) Applicant: METABOLON, INC., Morrisville, NC (US)

(72) Inventors: Rex A. DWYER, Raleigh, NC (US); Elizaveta FREINKMAN, Cary, NC (US); Anne M. EVANS, Cary, NC (US)

(21) Appl. No.: 18/684,128

(22) PCT Filed: Aug. 15, 2022
(86) PCT No.: PCT/IB2022/057633

§ 371 Date: Feb. 15, 2024

Related U.S. Application Data
(60) Provisional application No. 63/233,594, filed on Aug. 16, 2021.

(51) Int. Cl. G16C 20/20; G16C 20/70

(52) U.S. Cl. CPC G16C 20/20; G16C 20/70

(57) ABSTRACT

A method of structurally elucidating small molecule components, comprises determining, per sample, a molecular mass (MM) of a candidate compound (CC) fragment, determining possible molecular formulas (MF) having the fragment MM, and aggregating MS2 spectra for each CC fragment to form a candidate CC MS2 spectrum. Possible MFs and compound structures of an ion in the candidate MS2 spectrum consistent with the possible fragment MFs are determined. Known compounds (KC) similar via MS2 spectrum to the CC. KCs having a compound structure plausibly corresponding to the CC MS2 spectrum, a probability of the MS2 spectrum per fragment having compound substructures, and a combination of known fragment spectra (KFS) forming a compound spectrum statistically similar to the candidate MS2 spectrum of the CC, are determined. The possible MFs and compound structures. KCs, compound substructures, and combination of KFS, are associated with the MS2 spectrum of the CC/fragments thereof.

### BACKGROUND

### Field of the Disclosure

Aspects of the present disclosure relate to the analysis of small molecule components of a complex mixture and, more particularly, to a method and associated apparatus and computer program product for analyzing and elucidating the structure of small molecule components or compounds of a complex mixture, with such small molecule analysis including metabolomics, which is the study of small molecules produced by an organism's metabolic processes, or other analysis of small molecules produced through metabolism.

### Description of Related Prior Art

Compounds are diverse and numerous. The total number of compounds found in nature is unknown but is estimated to be at least in the tens of thousands. Ion data repositories (e.g., libraries) contain named compounds. Unnamed compounds are observed in biological samples but are not currently associated with library entries. Unnamed compounds may show significant correlations with disease, genetic variants, and other important biological metadata. As metabolomics data is collected on more and larger human cohorts, the number of biologically significant unnamed compounds is increasing. However, at present, the capability of elucidating the chemical structure of these unnamed compounds is a bottleneck that blocks the development of novel biomarkers, biological insights, and clinical interventions.

As currently practiced, structural elucidation of unnamed compounds is a laborious, time-consuming process that relies on manual examination of individual spectra and database searches driven by human pattern recognition and know-how. Specifically, although the exact mass of an unnamed compound can equate to a small number of candidate molecular formulas, publicly available databases contain either too few (e.g., zero) or far too many (e.g., hundreds) compound structures for each molecular formula. Therefore, in the current elucidation workflow, a human analyst must manually examine the liquid chromatography and tandem mass spectrometry (LC-MS1/MS2) data, at great length, and still be unable to propose a small number (e.g., 1-5) of testable structural compound candidates. That is, determining the identity and, ultimately, structure of a detected (but unnamed) compound is an important aspect of more thoroughly understanding the compound composition of a sample, but the current process of structural elucidation is manual and time-consuming.

As such, there exists a need for a method and associated apparatus and computer program product for analyzing and elucidating the structure of compound components or compounds of a complex mixture with increased speed and success rate compared to the manual process. It is also desirable to automatically predicting key structural features and stratifying structural candidates based on the LC-MS/MS characteristics of the unnamed compound.

### Definition of Terms

The terms “Small molecule”, “metabolite”, “compound” can be used interchangeably and mean organic and inorganic molecules which are present in a cell. The term does not include large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weights of over 2,000, 3,000, 4,000, 5,000, 6,000, 7,000, 8,000, 9,000, or 10,000). The small molecules of the cell are generally found free in solution in the cytoplasm or in other organelles, such as the mitochondria, where they form a pool of intermediates, which can be metabolized further or used to generate large molecules, called macromolecules. The term “small molecules” includes signaling molecules and intermediates in the chemical reactions that transform energy derived from food into usable forms. Non-limiting examples of small molecules include sugars, fatty acids, amino acids, nucleotides, intermediates formed during cellular processes, and other small molecules found within the cell.
The term “tandem MS” refers to an operation in which a first MS step, called the “primary MS” or “MS1”, is performed in a first mass spectrometer, followed by performance of one or more of a subsequent MS step, generically referred to as “secondary MS” or “MS2,” in the same (first) mass spectrometer or in a second mass spectrometer. In the primary MS (MS1), an ion, representing a chemical constituent, is detected and recorded during the creation of the primary mass spectrum. The substance represented by the ion is subjected to the secondary MS (MS2), in which the substance of interest undergoes fragmentation in order to cause the substance to break into sub-components, which are detected and recorded as a secondary mass spectrum. In a true tandem MS, there is an unambiguous relationship between the ion of interest in the primary MS and the resulting sub-component ions created during the secondary MS. The ion of interest in the primary MS corresponds to a “parent” or precursor ion, while the ions created during the secondary MS correspond to sub-components of the parent ion and are herein referred to as “daughter” or “product” ions. Tandem MS allows the creation of data structures that represent the parent-daughter relationship of chemical constituents in a complex mixture. This relationship can be represented by a tree-like structure illustrating the relationship of the parent and daughter ions to each other, where the daughter ions represent sub-components of the parent ion. Tandem MS can be repeated on daughter ions to determine “grand-daughter” ions, for example. Thus, tandem MS is not limited to two-levels of fragmentation but is used generically to refer to multi-level MS, also referred to as “MSⁿ”. The term “MS/MS” is a synonym for “MS2”.
RI (Retention Index)—a normalized measure of the retention time of a molecule in liquid chromatography.
SMILES (Simplified Molecular-Input Line-Entry System)—a standard system for representing the two-dimensional structure of a molecule by a string of characters. For example, Cl═CC(═CC═ClC(CC(═O)O)CN)Cl represents the two-dimensional structure of the drug baclofen. (Although some forms of SMILES include representation of stereochemistry, this disclosure refers to the form of SMILES, without representation of stereochemistry.)

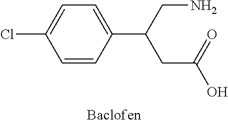 [SMILES: NCC(CC(=O)O)c1ccc(Cl)cc1]

Canonical SMILES—a “preferred” SMILES string to represent a given molecule. For example, both C(═O)═O and O═C═O represent carbon dioxide; however, the second form is “canonical”. Some software packages provide functions for putting SMILES strings into canonical form. To find if two strings represent the same molecule, each is “canonicalized” to see if the result is the same.
MS1 spectrum—MS1 sample data or MS1 sample components representing the chemical constituents detected and recorded at a given point in time during sample analysis.
Ion—a molecule with a net electric charge due to the gain or loss of subatomic particles (electrons or protons). Unless specified otherwise, in this disclosure, an ion refers to an MS2 ion.
Fragment—in this document, fragment refers exclusively to a fragment of the SMILES string of a molecule. SMILES fragments can be recognized by an asterisk ‘*’ representing the point at which the fragment has been broken off the molecule.
Exact mass—the theoretical mass of a molecule or species, found by summing the monoisotopic masses of its atomic constituents. Exact mass does not average over isotopic abundance in nature.
Accurate mass—the mass of a species as measured by mass spectrometers, which includes a mass error tolerance, e.g., exact mass ±5 ppm (parts per million).
MS2 spectrum—a mass spectrum or MS2 sample components produced by fragmenting a specific compound using a mass spectrometer, e.g., ThermoFisher's Orbitrap technology.
In silico fragmentation—The computational process of finding (SMILES) fragments of the SMILES string of a molecule.
SMARTS (SMiles ARbitrary Target Specification)—a language used for describing molecular patterns and properties in SMILES strings. For example, the SMARTS pattern [#16=SX2H0][#16=SX2H0] matches SMILES strings that describe disulfides.
Library—collection of proprietary information on compounds detected by a mass spectrometry based methodological process, including mass, RI, and MS fragmentation spectra of the compound including isotopes, in-source fragments and adducts. A library can also include public information such as SMILES strings, Inchi strings, InchiKey, etc. Examples and statistics in this disclosure refer to Library 209 (“NEG”) unless specified. “Library compounds” refers to compounds in a library.
Unnamed compound, unknown compound or X-compound—a compound known only by mass, RI, and MS2 spectrum; its chemical name, molecular formula, and chemical structure are unknown.
(Structural) elucidation—the process of determining the chemical structure and name of an unnamed compound.
LC—liquid chromatography
Persistent data structure—A table or other compilation of data saved on a storage device for repeated reference by the run-time software.

### SUMMARY

The above and other needs are met by aspects of the present disclosure which, in some aspects, provides a method and associated apparatus and computer program product for analyzing and elucidating the structure of small molecule components or compounds of a complex mixture, that determines the structure of new compounds in one or more samples in an automated manner that is faster and more accurate than existing manual methods. This is accomplished, in part, through tools and models built on large amounts of data from both an existing ion repository/chemical library and publicly available sources to quickly generate a list of arithmetically possible molecular formulas for a given molecular mass.

In some instances, the method and associated apparatus and computer program product for analyzing and elucidating the structure of small molecule components or compounds of a complex mixture involve:

1. for each unnamed compound, use precomputed tables to find molecular formulas for the mass of the unnamed compound that are arithmetically possible, satisfy double-bond constraints, are statistically similar to molecular formulas of known compounds, and satisfy isotopic constraints found during MS1 analysis:

2. aggregating the MS2 spectra stored for each unnamed compound to form a consensus MS2 spectrum:

3. annotating each ion of the consensus MS2 spectrum with a possible molecular formula. Using a precomputed table, find substructures observed in a library corresponding to those molecular formulas:

4. constructing a table showing consistency of ion molecular formulas with unnamed compound molecular formulas

5. finding the compounds in a library with MS2 spectra that are most similar to the consensus MS2 spectra of the unnamed compound according to a measure of spectrum-to-spectrum similarity:

6. searching a large private database and/or aggregating public database information for compounds with SMILES string that plausibly explain the MS2 spectrum using a measure of SMILES-to spectrum similarity:

7. using precomputed models, predicting the presence of various substructures expressed as SMILES strings from the MS2 spectrum of each unnamed compound:

8. attaching all of the above to stored representation of unnamed conpound for future reporting; and

9. Optionally, when desired, generating a graphical report of all of the above for the human elucidator, including color drawings of molecular structures.

Aspects of the present disclosure further provide:
1. A method using experimental fragmentation data and a novel machine-learning approach to propose candidate structures for the individual MS2 fragments of an unnamed compound:
2. Given the full MS2 fragmentation spectrum of an unnamed compound, a method using experimental fragmentation data and a second novel machine-learning approach to assess the likelihood that a particular candidate structure could give rise to that MS2 spectrum:
3. In performing the evaluation described in item #2,a method of searching through hundreds of thousands of candidate structures in large databases in a novel manner that is more rapid and efficient than existing methods; and
4. A method more accurate at determining the molecular formula of an unnamed compound than existing methods because, in addition to exact mass and isotopes, it uses the MS2 fragments as well as a novel metric called “harmony” to evaluate and rank the possible formulas.

One particular aspect of the present disclosure provides a method of analyzing data for one or more samples, the data for each sample being obtained from a component separation and tandem mass spectrometer system including a mass spectrometer conducting a first mass spectrometry step or function (MS1) and a second mass spectrometry step or function (MS2), and structurally elucidating small molecule components of the one or more samples. Such a method comprises, for each sample, determining a molecular mass of a fragment of a candidate compound, determining possible molecular formulas having the molecular mass of the fragment, and aggregating MS2 spectra for each of a plurality of fragments of the candidate compound to form a candidate MS2 spectrum of the candidate compound. Possible molecular formulas and compound structures of an ion in the candidate MS2 spectrum are determined, with the ion comprising one or more fragments, that are consistent with the possible molecular formulas of the fragments. Known compounds having an MS2 spectrum similar to the candidate MS2 spectrum of the candidate compound are determined, and known compounds having a compound structure plausibly corresponding to the MS2 spectrum of the candidate compound are determined. A probability of the MS2 spectrum of each fragment having one or more compound substructures is determined, and a combination of known fragment spectra forming a compound spectrum statistically similar to the candidate MS2 spectrum of the candidate compound is determined. The determined possible molecular formulas and compound structures, determined known compounds, determined compound substructures, and determined combination of known fragment spectra are then associated with the MS2 spectrum of the candidate compound and fragments thereof.

The present disclosure thus includes, without limitation, the following example embodiments:

Example Embodiment 1: A method of analyzing data for one or more samples, the data for each sample being obtained from a component separation and tandem mass spectrometer system including a mass spectrometer conducting a first mass spectrometry step or function (MS1) and a second mass spectrometry step or function (MS2), and structurally elucidating small molecule components of the one or more samples, said method comprising, for each sample, determining a molecular mass of a fragment of a candidate compound; determining possible molecular formulas having the molecular mass of the fragment; aggregating MS2 spectra for each of a plurality of fragments of the candidate compound to form a candidate MS2 spectrum of the candidate compound; determining possible molecular formulas and compound structures of an ion in the candidate MS2 spectrum, the ion comprising one or more fragments, that are consistent with the possible molecular formulas of the fragments; determining known compounds having an MS2 spectrum similar to the candidate MS2 spectrum of the candidate compound; determining known compounds having a compound structure plausibly corresponding to the MS2 spectrum of the candidate compound; determining a probability of the MS2 spectrum of each fragment having one or more compound substructures; determining a combination of known fragment spectra forming a compound spectrum statistically similar to the candidate MS2 spectrum of the candidate compound; and associating the determined possible molecular formulas and compound structures, determined known compounds, determined compound substructures, and determined combination of known fragment spectra with the MS2 spectrum of the candidate compound and fragments thereof.

Example Embodiment 2: The method of any preceding example embodiment, or combinations thereof, wherein determining possible molecular formulas having the molecular mass of the fragment, comprises determining arithmetically possible molecular formulas for the molecular mass of the fragment, with the arithmetically possible molecular formulas satisfying double-bond constraints, being statistically similar to molecular formulas of known metabolites, and satisfying isotopic constraints from MS1 analysis.

Example Embodiment 3: The method of any preceding example embodiment, or combinations thereof, wherein determining possible molecular formulas and compound structures of an ion in the candidate MS2 spectrum consistent with the possible molecular formulas of the fragments thereof, comprises determining possible isomeric substructures corresponding to the possible molecular formulas and compound structures of the ion.

Example Embodiment 4: The method of any preceding example embodiment, or combinations thereof, wherein determining known compounds having a compound structure plausibly corresponding to the MS2 spectrum of the candidate compound, comprises determining known compounds each having a SMILES string identifier plausibly corresponding to the MS2 spectrum of the candidate compound according to a measure of SMILES-to-spectrum similarity.

Example Embodiment 5: The method of any preceding example embodiment, or combinations thereof, determining known compounds having a compound structure plausibly corresponding to the MS2 spectrum of the candidate compound, comprises ranking plausible molecular formulas of the determined known compounds based on statistical similarity of the plausible molecular formulas to molecular formulas in a compound library.

Example Embodiment 6: The method of any preceding example embodiment, or combinations thereof, wherein determining a probability of the MS2 spectrum of each fragment having one or more compound substructures, comprises predicting whether one or more compound substructures, each expressed as a SMILES string, is present in the fragment from the MS2 spectrum of each fragment.

Example Embodiment 7: An apparatus for analyzing data for one or more samples, the data for each sample being obtained from a component separation and tandem mass spectrometer system including a mass spectrometer for conducting a first mass spectrometry step or function (MS1) and a second mass spectrometry step or function (MS2), the apparatus comprising a processor and a memory storing executable instructions that, in response to execution by the processor, cause the apparatus to at least perform the method steps of any preceding example embodiment, or combinations thereof.

Example Embodiment 8: A computer program product for analyzing data for one or more samples, the data for each sample being obtained from a component separation and tandem mass spectrometer system including a mass spectrometer for conducting a first mass spectrometry step or function (MS1) and a second mass spectrometry step or function (MS2), the computer program product comprising at least one non-transitory computer readable storage medium having computer-readable program code stored thereon, the computer-readable program code comprising program code for performing the method steps of any preceding example embodiment, or combinations thereof.

These and other features, aspects, and advantages of the present disclosure will be apparent from a reading of the following detailed description together with the accompanying drawings, which are briefly described below. The present disclosure includes any combination of two, three, four, or more features or elements set forth in this disclosure, regardless of whether such features or elements are expressly combined or otherwise recited in a specific embodiment description herein. This disclosure is intended to be read holistically such that any separable features or elements of the disclosure, in any of its aspects and embodiments, should be viewed as intended, namely to be combinable, unless the context of the disclosure clearly dictates otherwise.

It will be appreciated that the summary herein is provided merely for purposes of summarizing some example aspects so as to provide a basic understanding of the disclosure. As such, it will be appreciated that the above described example aspects are merely examples and should not be construed to narrow the scope or spirit of the disclosure in any way. It will be appreciated that the scope of the disclosure encompasses many potential aspects, some of which will be further described below, in addition to those herein summarized. Further, other aspects and advantages of such aspects disclosed herein will become apparent from the following detailed description taken in conjunction with the accompanying drawings which illustrate, by way of example, the principles of the described aspects.

### BRIEF DESCRIPTION OF THE SEVERAL VIEWS OF THE DRAWING(S)

Having thus described the disclosure in general terms, reference will now be made to the accompanying drawings, which are not necessarily drawn to scale, and wherein:

FIG. 1 schematically illustrates a system according to one aspect of the present disclosure including a memory device having a database, a processor device, and a user interface (display), in communication with a spectrometry device;

FIG. 2 schematically illustrates a three-dimensional plot of spectrometry data associated with one exemplary sample:

FIG. 3 schematically illustrates a two-dimensional profile plot for one exemplary sample that may be determined from the corresponding three-dimensional plot of spectrometry data for that sample according to some aspects of the present disclosure:

FIG. 4 schematically illustrates a two-dimensional profile plot for one exemplary sample that may be determined from the corresponding three-dimensional plot of spectrometry data for that sample according to some aspects of the present disclosure; and

FIG. 5 schematically illustrates a method of analyzing, discerning, and structurally elucidating small molecule components or compounds of a complex mixture, according to one example aspect of the present disclosure.

### DETAILED DESCRIPTION OF THE DISCLOSURE

The present disclosure now will be described more fully hereinafter with reference to the accompanying drawings, in which some, but not all aspects of the disclosure are shown. Indeed, the disclosure may be embodied in many different forms and should not be construed as limited to the aspects set forth herein; rather, these aspects are provided so that this disclosure will satisfy applicable legal requirements. Like numbers refer to like elements throughout.

The various aspects of the present disclosure mentioned above, as well as many other aspects of the disclosure, are described in further detail herein. The apparatuses, methods, and computer program products associated with aspects of the present disclosure are exemplarily disclosed, in some instances, in conjunction with an appropriate analytical device which may, in some instances, comprise a separator portion or separation portion (e.g., a chromatograph) and/or a detector portion (e.g., a spectrometer). One skilled in the art will appreciate, however, that such disclosure is for exemplary purposes only to illustrate the implementation of various aspects of the present disclosure. Particularly, the apparatuses, methods, and computer program products associated with aspects of the present disclosure can be adapted to any number of processes that are used to generate complex sets of data for each sample (e.g., within a single sample), or over/across a plurality of samples, whether biological, chemical, or biochemical, in nature. For example, aspects of the present disclosure may be used with and applied to a variety of different analytical devices and processes including, but not limited to: analytical devices including a separator portion (or “component separator” or “component separation” portion) comprising a liquid chromatograph (LC), a gas chromatograph (GC), a supercritical fluid chromatograph (SFC), a capillary electrophoresis (CE) analyzer; a cooperating detector portion (or “mass spectrometer” portion) comprising a mass spectrometer (MS); an ion mobility spectrometry mass spectrometer (IMS-MS); and an electrochemical array (EC); and/or combinations thereof (e.g., a tandem mass spectrometer including MS1 and MS2 functionality). In some aspects of the present disclosure, the detector portion may be used without a separator portion.

In this regard, one skilled in the art will appreciate that the aspects of the present disclosure as disclosed herein are not limited to metabolomics analysis. For example, the aspects of the present disclosure as disclosed herein can be implemented in other applications where there is a need to characterize or analyze small molecules present within a sample or complex mixture, regardless of the origin of the sample or complex mixture. For instance, the aspects of the present disclosure as disclosed herein can also be implemented in a bioprocess optimization procedure where the goal is to grow cells to produce drugs or additives, or in a drug metabolite profiling procedure where the goal is to identify all metabolites that are the result of biotranformations of an administered xenobiotic. Some other non-limiting examples of other applications could include a quality assurance procedure for consumer product manufacturing where the goal may be to objectively ensure that desired product characteristics are met, in procedures where a large number of sample components can give rise to a particular attribute, such as taste or flavor (e.g., cheese, wine or beer), or scent/smell (e.g., fragrances). One common theme thus exhibited by the aspects of the present disclosure as disclosed herein is that the small molecules in the sample can be analyzed using the various apparatus, method and computer program product aspects disclosed herein.

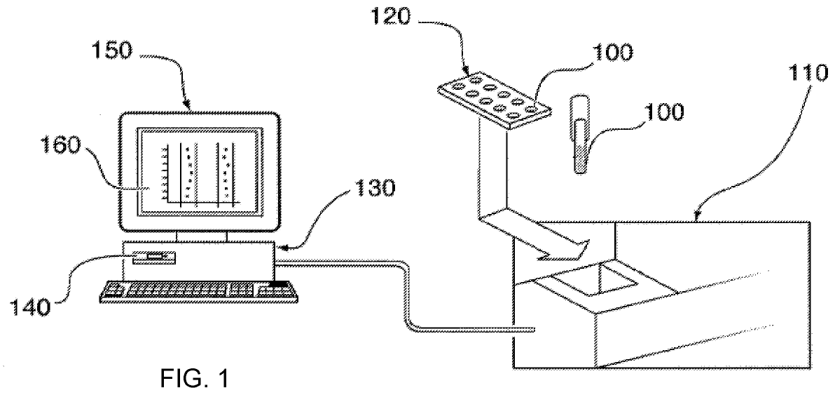

FIG. 1 illustrates an example of a system according to one aspect of the present disclosure wherein the system is in communication with an analytical device 110, such as a combination chromatograph (component separator/component separation)/tandem mass spectrometer (MS1, MS2). One skilled in the art will appreciate, however, that the configurations of an analytical device 110 presented herein are for exemplary purposes only, and are not intended to be limiting with respect to the scope of suitable and appropriate analytical devices that may also be applied under the principles disclosed herein. As shown, a sample (whether biological, chemical, or biochemical, in nature) 100 may be introduced into the separator portion/separation portion of the analytical device 110 and analyzed using appropriate techniques, as applied through the first mass spectrometer process/functionality (MS1) and the second mass spectrometer process/functionality (MS2) of the detector portion (wherein MS1 and MS2 implement the same mass spectrometer or different mass spectrometers), that will be appreciated by those skilled in the art.

For example, the components of a particular sample 100 may pass through a column associated with the separator portion/separation portion, at different rates and exhibit different spectral responses (e.g., associated with intensity as a function of retention time), as detected by the first mass spectrometer functionality (MS1) of the detector portion, based upon their specific characteristics. The second mass spectrometer functionality (MS2) adds a second phase of mass fragmentation which may be implemented, for example, to facilitate quantitation of low levels of compounds in the presence of a high sample matrix background. As will be appreciated by one skilled in the art, the analytical device 110 may generate a set of spectrometry data, corresponding to each sample 100 and having three or more dimensions (e.g., quantifiable samples properties) associated therewith, wherein the data included in the data set generally indicates the composition (e.g., sample components) of the sample 100. In some aspects, the data set may comprise, for example, data for each sample related to retention time, sample or component (ion) mass, intensity, or even sample indicia or identity. However, such data must first be appropriately analyzed in order to determine the sample composition (e.g., ions, metabolites).

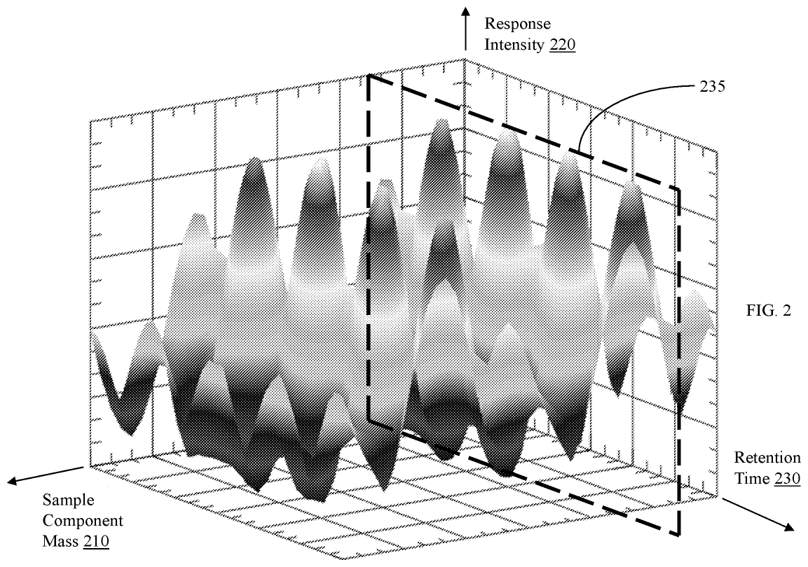

In some instances, a three-dimensional data set (MS1 or MS2) for each of one or more samples may be selected or otherwise designated for further analysis, with each dimension corresponding to a quantifiable sample property. An example of such a three-dimensional set of spectrometry data is shown generally in FIG. 2, and may be plotted on a three-axis plot or graph, with the plot or graph including individual axes for a response intensity element 220, a sample component mass element 210, and a time element 230 (particularly, in this example, the retention time or the time that a particular component spends in the column of the separator portion of the analytical device 110). That is, the data obtained for a particular sample, in some aspects, includes a relationship between ion mass 210, retention time 230, and intensity 220, including intensity 220 as a function of retention time 230 for a particular ion mass 210. The location of data points in relation to the sample component mass axis 210 may be indicative, for example, of the number of individual component molecules within the sample 100 and the relative mass values for such sample components.

According to other aspects of the present disclosure, different analytical devices may be used to generate a three or more dimensional set of analytical data corresponding to the sample 100. For example, the analytical device may include, but is not limited to: various combinations of a separator portion/separation portion comprising one of a liquid chromatograph (LC) (positive or negative channel) and a gas chromatograph (GC), a supercritical fluid chromatograph (SFC), a capillary electrophoresis (CE) analyzer; and a cooperating detector portion comprising one of a mass spectrometer (MS); an ion mobility spectrometer (IMS), a tandem mass spectrometer (MS1 and MS2); and an electrochemical array (EC). In some aspects, the analytical device may include a detector portion without a separator portion. One skilled in the art will appreciate that such complex three or more dimensional data sets may be generated by other appropriate analytical devices that may be in communication with components of aspects of the present disclosure as described in further detail herein.

One or more samples 100 may be taken individually from a well plate 120 and/or from other types of sample containers and introduced individually into the analytical device 110 for analysis and generation of the corresponding three or more dimensional data set (see, e.g., FIG. 2). For example, individual samples 100 may be transferred from a well plate 120 to the analytical device 110 via pipette, syringe, microfluidic passageways defined by a test array, and/or other systems for transferring samples in a laboratory environment. As disclosed herein, the nature of the samples may vary considerably, generally comprising mixtures or complex mixtures including small molecules, wherein such samples may exemplarily include, but are not limited to: blood samples, urine samples, cell cultures, saliva samples, plant tissue and organs (e.g., leaves, roots, stems, flowers, etc.), plant extracts, culture media, membranes, cellular compartments/organelles, cerebral spinal fluid (CSF), milk, soda products, food products (e.g., yogurt, chocolate, juice), and/or other types of biological, chemical, and/or biochemical samples in which the metabolites and/or chemical/molecular components of interest may be present. Of these possible samples or sample types, one common aspect is that the selected sample includes a known characteristic. This known characteristic may be, for example, at least a general type or classification, a source, etc. Empirical data or other information associated with the known characteristic of the sample may be implemented to determine, for example, one or more ions, small molecules or metabolites expected to be present in such a sample having that known characteristic. That is, such information associated with the known characteristic provides a context to the sample and the data obtained therefrom via the component separation and mass spectrometer system, wherein the context provides an indicium or indicia at least as to a basic component or constituent of the sample.

As shown in FIG. 1, aspects of the present disclosure may comprise an ion data repository (e.g., a library) comprising, for example, a database (e.g., a relational database) stored at least in part, for example, as executable or accessible instructions in a memory or memory device 140 (i.e., a computer-readable storage medium having computer-readable program code portions stored therein), wherein the memory device 140 is in communication with a processor or processor device 130 (e.g., a computer device implementing a processor) for selectively executing the instructions/computer-readable program code portions in the memory device 140 to cause an apparatus to perform particular method steps and/or functions. In some instances, the memory device 140 and/or the processor device 130 may be configured to be in communication, whether directly or indirectly, with the analytical device 110 for receiving a data set (in some instances, a data set comprising three or more dimensions, wherein a data parameter such as sample indicia, sample or component (ion) mass, retention time, and intensity/response may represent any one of the dimensions of the data set), corresponding to the sample 100, therefrom. That is, the dataset received by the memory device includes, for example, data indicating a relationship between ion mass, retention time, and intensity. In some particular instances, the dataset (for each of one or more samples 100) includes data indicating intensity as a function of retention time for a particular ion mass. The processor device 130 may be in communication with the analytical device 110 via wire line (RS-232, and/or other types of wire connection) and/or wireless (such as, for example, RF, IR, or other wireless communication) techniques such that the database associated with the memory device 140/processor device 130 (and/or in communication therewith) may receive the data set from the analytical device 110 so as to be stored thereby.

Furthermore, the analytical device 110 may be in communication with one or more processor devices 130 (and associated user interfaces and/or displays 150) via a wire line and/or wireless computer network including, but not limited to: the Internet, local area networks (LAN), wide area networks (WAN), or other networking types and/or techniques that will be appreciated by one skilled in the art. The user interface/display 150 may be used to receive user input and to convey output such as, for example, displaying any or all of the communications involving the system, including the manipulations and analyses of sample data disclosed herein, as will be understood and appreciated by one skilled in the art. The database may be structured using commercially available software, such as, for example, Oracle, Sybase, DB2, or other database software. As shown in FIG. 1, the processor device 130 may be in communication with the user interface/display 150 and the memory device 140 (such as a hard drive, memory chip, flash memory, RAM module, ROM module, and/or other memory device 140) for storing/administering the ion data repository/database, including the data sets received from the analytical device 110, whether automatically (directly) or indirectly. In addition, the memory device 140 may also be used to store other received data or information involving the sample(s) or component(s) thereof in the ion data repository/database and/or data otherwise manipulated by the processor device 130.

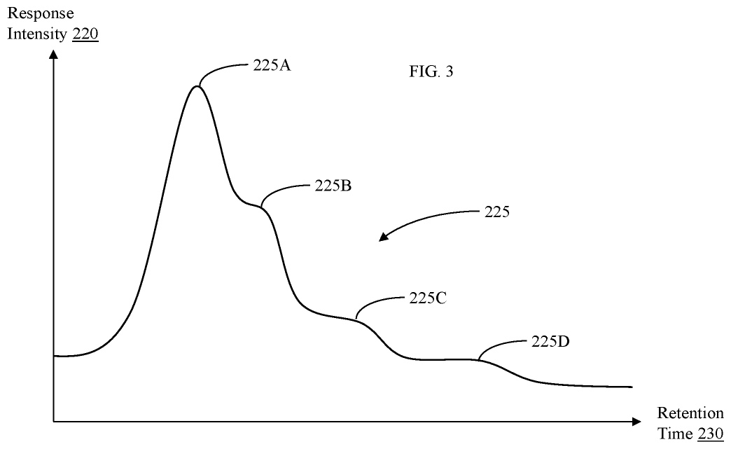

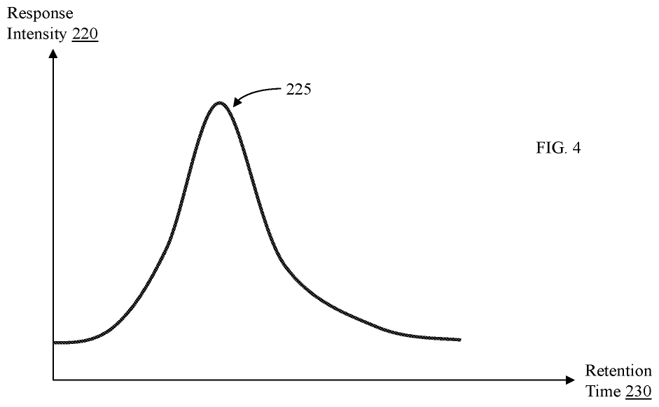

The processor device 130 may, in some aspects, be capable of converting each of the data sets, each including, for example, data indicating a relationship between various sample parameters such as ion mass, retention time, and intensity (see, e.g., FIG. 2, wherein the exemplary data set is a three-dimensional data set) for each of the samples, received by the memory device 140, into at least one corresponding two-dimensional data set (see, e.g., FIG. 3). The at least one two-dimensional data set may comprise, for example, a two-dimensional component “profile” of a particular sample 100 at a particular point 235 (FIG. 2) along one of the three axes of the three-dimensional data set. The particular point 235 along one of the three axes may be, for example, a particular selected sample component mass along the sample component mass axis 210. Once that particular sample component mass is selected, the resulting “slice” of the three-dimensional data set becomes the two-dimensional profile plot for the sample. That is, the resulting profile (also referred to herein as a “profile plot” as shown in FIGS. 3 and 4) illustrates that particular sample component mass detected (and the intensity of that detection) as a function of time measured from a zero point, the zero point corresponding to when the sample 100 is injected and/or otherwise introduced into the analytical device 110). For example, the processor device 130 may be configured to produce a detection intensity/response versus/as a function of sample component (retention) time two-dimensional profile of the sample for that given or selected sample component mass point 235 (see FIGS. 3 and 4, for example). The “x” axis in FIG. 2 (or (retention) time axis 230, for example) may further, in some instances, be characterized as a retention index (e.g., the retention time of an ion/compound normalized to the retention times of adjacently eluting known ions/compounds) and/or a retention time. Thus, the processor device 130 may be further capable of parsing each of the three (or more) dimensional data sets, for each of the plurality of samples, into one or more individual two-dimensional (i.e., intensity/response versus sample component retention time profile) profiles corresponding to at least one particular (selected) sample component mass point (element 235, for example) so as to convert each three (or more) dimensional data set (of FIG. 2, for example) into at least one corresponding two-dimensional data set of a selected sample component (having a profile or profile plot shown, for example, in FIGS. 3 and 4) that may further be plotted as an response intensity 220 of the corresponding sample component mass versus a sample component retention time 230 (or retention index), and displayed on the user interface/display 150, as desired. One skilled in the art will appreciate that any amount of two-dimensional data sets or profile plots may be formed or obtained from any three or more dimensional data sets by selecting two different sample parameters at a selected particular value of a third sample parameter, and then plotting the two different sample parameters against each other in a two-dimensional plot.

According to some aspects, the processor device 130 may be configured to selectively execute the executable instructions/computer-readable program code portions stored by the memory device 140, if necessary, in cooperation with the ion data repository/library/database also stored by the memory device 140, so as to accomplish, for instance, the identification, quantification, representation, curation, and/or other analysis of a selected sample component (i.e., a metabolite, molecule, or ion, or portion thereof) in each of the plurality of samples (or within a single sample), from the two-dimensional data set representing the respective sample among the plurality of samples. In doing so, the sample component of interest from the sample to be analyzed is first determined from at least one known characteristic associated with the sample. The at least one known characteristic associated with the sample may include, for example, at least a general type or classification, a source, etc. In some aspects, the at least one known characteristic may involve a particular nature of the sample, wherein the particular nature of the sample may vary considerably, from generally comprising mixtures or complex mixtures including small molecules, to particularly and exemplarily including, without limitation: blood samples, urine samples, cell cultures, saliva samples, plant tissue and organs (e.g., leaves, roots, stems, flowers, etc.), plant extracts, culture media, membranes, cellular compartments/organelles, cerebral spinal fluid (CSF), milk, soda products, food products (e.g., yogurt, chocolate, juice), and/or other types of biological, chemical, and/or biochemical samples. The at least one known characteristic, in particular aspects, indicates which metabolites and/or chemical/molecular components of interest may be present in that sample (or which metabolites and/or chemical/molecular components which are not expected to be in the sample). That is, in addition to data regarding discrete particular ions, the ion data repository/library/database may also include empirical data or other information associated with the known characteristic of the sample.

Accordingly, upon identifying the at least one known characteristic of the sample or receiving the at least one known characteristic as an input via the user interface/display 150, the processor 130 may be configured to execute computer-readable program code portions stored by the memory device 140 for implementing the empirical data and other information to correlate the one or more known characteristics with one or more particular ions, small molecules or metabolites expected to be present in such a sample having that known characteristic. That is, in some aspects, such information and empirical data associated with the one or more known characteristics provides a context to the sample and the data obtained therefrom, wherein the context provides an indicium at least as to a basic component or constituent of the sample, or where relevant data may be located within the ion data repository/database. In turn, the particular identifying data associated with the indicium of the basic component or constituent, or information location within the ion data repository/database, further indicates candidate ions, compounds and components that may be present or are expected or predicted to be present in the sample under analysis. That is, in particular aspects, comparing the known characteristic to empirical data included in the ion data repository, wherein the empirical data includes relational information between known characteristics and certain ions, allows the determination therefrom of the one or more ions corresponding to the known characteristic(s). In particular aspects of the disclosure, the selecting, based on the known characteristic, of one or more ions from the ion data repository expected to be included in the sample may be facilitated by more extensive information and empirical data received and housed within the ion data repository/database, wherein any “learning” by the processor 130 represents efficiencies and accuracies gained from additional correlative information.

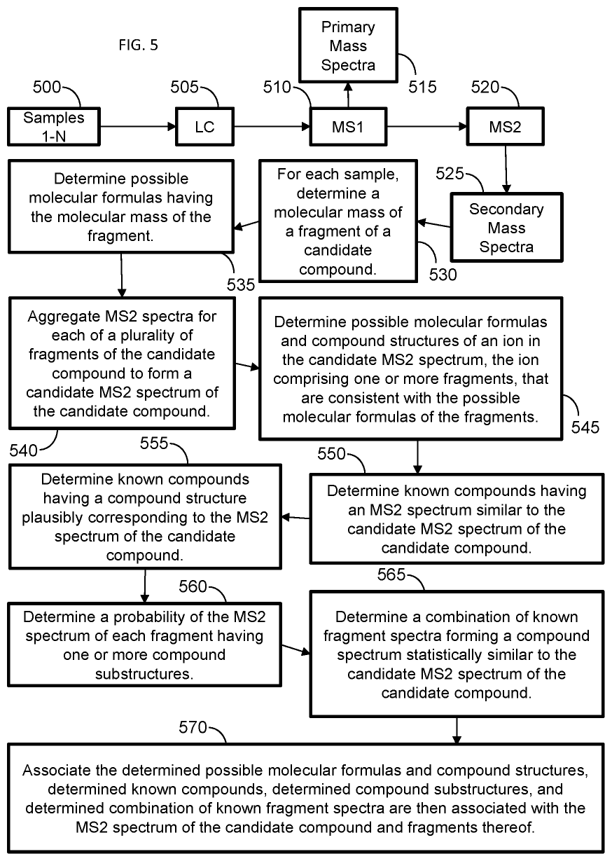

According to particular aspects, as shown in FIG. 5, a method of analyzing data for one or more samples (Block 500) and structurally elucidating small molecule components of the one or more samples is provided, with the data for each sample being obtained from a component separation and tandem mass spectrometer system comprising a separation portion (Block 505), including a liquid chromatograph, a gas chromatograph, a supercritical fluid chromatograph, or a capillary electrophoresis analyzer, and a first mass spectrometry step or provision (MS1) (Block 510) and a second mass spectrometry step or provision (MS2) (Block 515), wherein the data from the MS1 includes MS1 sample components (primary mass spectra-Block 520) and the data from the MS2 includes MS2 sample components (secondary mass spectra-Block 525).

Such a method comprises, for each sample, determining a molecular mass of a fragment of a candidate compound (Block 530); determining possible molecular formulas having the molecular mass of the fragment (Block 535); and aggregating MS2 spectra for each of a plurality of fragments of the candidate compound to form a candidate MS2 spectrum of the candidate compound (Block 540). Possible molecular formulas and compound structures of an ion in the candidate MS2 spectrum, the ion comprising one or more fragments, are determined that are consistent with the possible molecular formulas of the fragments (Block 545), and known compounds having an MS2 spectrum similar to the candidate MS2 spectrum of the candidate compound are also determined (Block 550). In further aspects, known compounds having a compound structure plausibly corresponding to the MS2 spectrum of the candidate compound are determined (Block 555), a probability of the MS2 spectrum of each fragment having one or more compound substructures is determined (Block 560), and a combination of known fragment spectra forming a compound spectrum statistically similar to the candidate MS2 spectrum of the candidate compound is determined (Block 565). The determined possible molecular formulas and compound structures, determined known compounds, determined compound substructures, and determined combination of known fragment spectra are then associated with the MS2 spectrum of the candidate compound and fragments thereof (Block 570).

Further aspects of the methods, apparatuses, and computer program products disclosed herein are as follows:

1. A criterion (harmony) for identifying plausible molecular formulas based on statistical similarity to molecular formulas in a compound library.

A formula that is arithmetically consistent with a given accurate mass may still not be chemically possible. Assessing “chemical possibility” computationally is difficult, requiring analysis of volumes of atoms, distribution of charges, etc. The goal is to determine the molecular formula of metabolites specifically by asking the question of interest: “Is this formula plausibly the formula of a metabolite?”

In instances where the formulas of (e.g., several thousand) metabolites, wherein the formulas of many more metabolites could be retrieved from public databases, the question can be rephrased: Is this formula like the formulas of compounds that can currently be detected? In statistical learning theory, this is the Anomaly Detection Problem.

One goal is to assign a number to any formula that is relatively high in plausibility for formulas already in the library, and low for formulas that are not seemingly plausible. As an example, C₁₈H₃₅N₂OPS is arithmetically consistent with exact mass 358.2221 u, but may not necessarily correlate to the metabolites that can be or have been detected (e.g., in context). The number assigned to C₁₈H₃₅N₂OPS by the process described below is thus 1 (e.g., on a scale from 0 to 100), wherein the assigned number represents the harmony of that formula.

To use any anomaly detection algorithm, it will be necessary to define the “features” of every formula. An effective set of features (with values for the example C₁₈H₃₅N₂OPS, exact mass 358.2208 u), in one example, could be:
Fraction of atoms that are carbon: 18/58=0.313
Fraction of atoms that are hydrogen: 35/58=0.603
Fraction of atoms that are nitrogen: 2/58=0.034
Fraction of atoms that are oxygen: 1/58=0.017
Fraction of mass constituted by carbon: 18*12/358.2208=0.603
Fraction of mass constituted by hydrogen: 35*1.0078/358.2208=0.098
Fraction of mass constituted by nitrogen: 2*14.0031/358.2208=0.078
Fraction of mass constituted by oxygen: 15.9949/358.2208=0.045

Any of a number of published anomaly detection algorithms could be applied, but the one chosen to exemplify the method is Local Outlier Factors. (Breunig, M. M.; Kriegel, H.-P.; Ng, R. T.; Sander, J. (2000) LOF: Identifying Density-based Local Outliers (PDF). Proceedings of the 2000 ACM SIGMOD International Conference on Management of Data. SIGMOD. pp. 93-104. doi: 10.1145/335191.335388. ISBN 1-58113-217-4.).

The inputs to the process are (a) the features of all the entries in the library and (b) the features of the formula to be assessed as a possible anomaly. The methodology begins with a “training” phase on the library entries' features (a), which can be carried out once for all subsequent use (such a process may take, for example, on the order of a few minutes). The training phase assigns a local outlier factor (LOF) to every formula in the library. Formulas with lower LOFs are identified as “unusual”. The distribution of LOFs for the library entries has a long lower tail.

The LOF for the example formula C₁₈H₃₅N₂NOPS is −0.645, putting it among the lowest 1% of library formulas, and its harmony is reported as 1. Another formula for the same mass, C₁₆H₃₀N₄O₅, has an LOF of 0.228 at the 59^{th} percentile, harmony=59. Other evidence (e.g., context) also indicates that C₁₆H₃₀N₄O₅ is the correct formula for the feature detected at this mass.

The training phase may be time-consuming, and it is desirable in any instance to precompute the statistical model once so as to give consistent results across runs of the program. Therefore, the results of the training phase are saved in a persistent form (for example, Python pickle files), and the harmony of each query formula is quickly computed using the saved statistical model.

If the concept herein for identifying metabolites is expanded to identifying formulas that are plausibly drugs or plausibly in another category, the same analysis principles herein will apply.

2. MS2SMILES criterion for assessing similarity of the MS/MS fragmentation spectrum of an unknown molecule to the structure of a known molecule as represented by a SMILES string.

To search for similarities between X-compounds with known spectra but unknown SMILES string, and known compounds with SMILES strings but no spectra in a database, it is useful to have a measure of similarity between a spectrum and a SMILES string, which has been termed MS2SMILES.

A SMILES string can be broken into fragments, wherein each fragment has an exact mass, but no intensity. On the other hand, a spectrum has observed accurate masses and measured intensities. If the spectrum and SMILES string correspond, it is expected that some of the spectral masses will match some of the fragment masses. However, some fragments are never directly represented in any analyzed spectrum, and many spectral masses arise from processes other than the simple breakage of a single covalent bond.

To measure the similarity of a spectrum to a SMILES string, the subset of the spectral masses that match (e.g., +5 ppm) fragment masses are determined and termed the assigned masses. The intensities of the assigned masses are them summed to provide a raw score.

If comparing a single spectrum to a plurality of SMILES strings, it may suffice to compare raw scores to find the “best” match, but the raw score itself gives little indication of absolute quality of the match. It would thus be helpful to know what fraction of all intensities has been assigned to fragment masses. However, this number will be diluted by spectral masses that can't ever arise from any single-bond-breakage process in any plausible molecule; roughly ⅓ of the masses in the spectra in the library fall into this category. These masses are effectively unassignable in any circumstance. To identify them requires a list of assignable exact masses. Given a large database of SMILES strings, all can be fragmented to find a large set of fragments, and the masses of these fragments are the assignable exact masses.

The final MS2SMILES score is:

(sum of intensities of assigned masses)/(sum of intensities of assignable masses)

As an example, here is the matching of spectrum X-21821 (an X-compound) and the SMILES string of indolyl-3-acryloylglycine (IAG):

### Fragmentation of IAG: O═C(O)CNC(═O)C═Cc1c[nH]c2ccccc12

| FRAGMENT | MASS |
|---|---|
| *O | 17.0032 |
| *C(═O)O | 44.9982 |
| *CC(═O)O | 59.0138 |
| *NCC(═O)O | 74.0247 |
| *C(═O)NCC(═O)O | 102.0196 |
| *c1c[nH]c2ccccc12 | 116.0505 |
| *C═CC(═O)NCC(═O)O | 128.0353 |
| *C═Cclc[nH]c2ccccc12 | 142.0662 |
| *C(═O)C═Cc1c[nH]c2ccccc12 | 170.0611 |
| *NC(═O)C═Cc1c[nH]c2ccccc12 | 185.0720 |
| *CNC(═O)C═Cc1c[nH]c2ccccc12 | 199.0876 |
| *C(═O)CNC(═O)C═Cc1c[nH]c2ccccc12 | 227.0826 |

The MS2 spectrum of X-21821 appears in tabular form in the left two columns of the Table below. Three masses (74, 117, 143) do not correspond to any known fragments for any compound in the database, so these intensities are not assignable in column 3. Of the 12 assignable masses, only three can be assigned to the fragments of LAG. The total assigned intensity is 138.6, and the total assignable intensity is 166.4. The MS2SMILES score is 0.833, a very high score (since 1 is a “perfect score”.) X-21821 was confirmed to be LAG. The structure of LAG is shown below.

| spec_mass | intensity | assignable | assigned | fragment |
|---|---|---|---|---|
| 74.0254 | 4.6 | | | |
| 82.0296 | 6 | 6 | | |
| 100.0039 | 5.3 | 5.3 | | |
| 116.0505 | 37.1 | 37.1 | 37.1 | *c1c[nH]c2ccccc12 |
| 117.0539 | 2.8 | | | |
| 130.0663 | 1.2 | 1.2 | | |
| 140.0507 | 4.8 | 4.8 | | |
| 142.0663 | 100 | 100 | 100 | *C═Cc1c[nH]c2ccccc12 |
| 143.0699 | 9.7 | | | |
| 144.0819 | 1.3 | 1.3 | | |
| 156.082 | 4 | 4 | | |
| 157.0766 | 1.2 | 1.2 | | |
| 168.0457 | 2.8 | 2.8 | | |
| 169.0774 | 1.2 | 1.2 | | |
| 170.0613 | 1.5 | 1.5 | 1.5 | *C(═O)C═Cc1c[nH]c2ccccc12 |

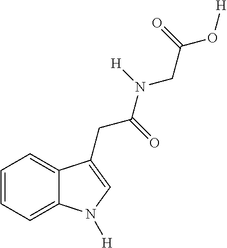 [SMILES: O=C(O)CNC(=O)Cc1c[nH]c2ccccc12]

It is generally expected that many fragments are not necessarily matched by ions. As such, it is not anticipated that every fragment found theoretically by in silico fragmentation will be realized during mass spectrometry. On the other hand, many assignable ions are not assigned. This is likely because the same accurate mass can be generated by both single-bond-breaking and other processes. Another possible source of ions is another compound that contaminated the MS2 of the compound of interest. Since the ions that were assigned in the example included the two largest ions, the score is still high. The fact that one of the ions also matches a very large fragment of the SMILES string for IAG, and a large fraction of its mass also supports the hypothesis that the spectrum does correspond to IAG. On the other hand, for example, the spectral mass 116.0505 could be any fragment with molecular formula CoHN (or other formulas of similar mass).

To search for similarities between unnamed compounds with known spectra but unknown SMILES string, and known compounds with SMILES strings but no spectra in a database, it is useful to have the MS2SMILES measure of similarity between a spectrum and a SMILES string. This matching involves, for example, matching matches and losses in the observed spectrum to matches and losses that could potentially occur in the compound represented by the SMILES string when subjected to fragmentation in a mass spectrometer. There is no expectation that every potential ion or loss suggested for a SMILES string will be found in the observed spectrum. There is generally no attempt to predict a complete spectrum with intensities; rather, only the presence or absence of an ion/loss is of interest.

Predictions can be generated in two ways:
A. A deterministic method: A SMILES string can be broken into fragments at all single covalent bonds in silico. Each fragment has an exact mass, but no intensity. On the other hand, a spectrum has observed accurate masses and measured intensities. If the spectrum and SMILES string correspond, some of the spectral masses are expected to match some of the fragment masses.
B. A statistical method: An existing library of known compounds contains both observed spectra and SMILES strings. An ion or loss that occurs in a spectrum will also occur in many other spectra. Likewise, the SMILES strings can be fragmented in silico, and most fragments will occur in many compounds. Therefore, a machine learning model is trained for each ion and loss in which the predictive variables are 0/1 variables, recording the presence or absence of a SMILES fragment in a known compound. More specifically, each row of the training matrix represents a single compound, and each column represents a single possible SMILES fragment. The entry for a particular known compound row and fragment column is 1 if the fragment occurs in the fragmentation of the SMILES string of the known compound and 0 otherwise. To create a model for a particular observed ion or loss, a response vector is created with an entry for each known compound that is 1 if the ion (or loss) is present in the observed spectrum for the known compound and 0 otherwise. Then a statistical classification model can be trained that can predict the presence of the ion (or loss) in the spectrum of a known compound given only the SMILES string of the known compound. There will be thousands of such models predicting the thousands of distinct ions (or losses) observed in the totality of the observed spectra of a reference library. These models can be created by many machine learning methods such as Neural Nets, Random Forests, or Support Vector Machines, but in this disclosure, Logistic Regression has been employed.

Given a SMILES string, an observed spectrum, and the set of models, the compatibility of the SMILES string with the spectrum can be assessed by deterministically suggesting ions (and losses) from the SMILES string according to A and then statistically predicting ions and losses for the SMILES strings according to B. Then the match is evaluated by looking at what “fraction” of the observed spectrum is accounted for by the suggestions and predictions for the SMILES strings. In such an aspect, that “fraction” is defined as ratio of the summed intensities of the observed ions matched by the SMILES string divided by the sum of the intensities of all observed ions.

In general, there may be thousands of models for observed ions. Evaluating each for a SMILES string could require an impractical amount of computations if carried out for many SMILES strings. However, this is unnecessary when comparing a single spectrum because at most only a few dozen models are of interest, namely the models for the ions (and losses) actually observed.

3. A methodology for quickly searching for all compounds similar to a fragmentation spectrum in a large (>100,000 entries) database of compounds represented by SMILES strings (RapidSMatch) using the MS2SMILES criterion.

With a large database of compounds with SMILES assembled from public sources, it is desirable to be able to search the database quickly for all compounds similar to a single query spectrum of an X-compound according to the MS2SMILES criterion, since the compounds with the highest scores will be candidate structures for the X-compound. It is not preferred to search compound by compound mainly because each SMILES string in the database would need to be fragmented for each query. Fragmentation is a processor-intensive operation. It is preferable to pre-compute fragmentations once and save those fragmentations in a computer file storage format (e.g., disk file) or other persistent data format. Once fragmentation is completed, other chemical structures enabling fast processing of query spectra can be precomputed and saved as well.

### Data Structures:

A. “Data Structure A” is a data structure for each fragment mass, mapping that (exact) mass to a list of SMILES strings from the database that contain a fragment with that mass. This data structure should be indexed in some manner by fragment mass. Of the many possibilities, including, for example, Binary Search, Digital Binning, Hash Tables with internal Chaining, Hash Tables with External Chaining, Organ-Pipe Hash Tables, Robin Hood Hash Tables, Red-Black Trees, AVL Trees, and Skip Lists, a sorted list supporting binary search was chosen for the exemplification of the methodology herein.
B. “Data Structure B” is a data structure indexed by fragment mass mapping accurate masses to the matching lists of Data Structure A above. Of the many possibilities, including, for example, Binary Search, Digital Binning, Hash Tables with internal Chaining, Hash Tables with External Chaining, Organ-Pipe Hash Tables, Robin Hood Hash Tables, Red-Black Trees, AVL Trees, and Skip Lists, a binning method was chosen for the exemplification herein.
C. “Data Structure C” is a data structure constructed for each query for accumulating MS2SMILES assigned intensities for each of a subset of the database SMILES strings. This data structure includes a floating-point number for each SMILES string, and it is indexed by SMILES string. For exemplification herein, a hashing index (e.g., Python built-in dictionary) was chosen.

### Precomputation—a One-Time Table-Building Process

For each SMILES string in the database:
1. Fragment the string.
2. Determine the exact masses of the fragments.
3. For each fragment mass:
   a. Query Data Structure B to locate the correct Data Structure A for the fragment mass.
   b. Insert the SMILES into the correct Data Structure A

### Query—Repeatedly at Run Time

1. Create a Data Structure C for the query.
2. For each (accurate mass, intensity) pair in the query spectrum:
   a) Using Data Structure B, find all fragment masses (exact masses) to which the accurate mass can be assigned, if any.
   b) Using Data Structure A for each of the fragment masses, find the set of SMILES strings in the desired mass range for the query results.
   c) Find the union of the sets from the previous step.
   d) For each SMILES string in the union, add the intensity to its accumulating assigned intensity in Data Structure C.
   e) Add the intensity to the accumulating assignable intensity.
3. For each SMILES string in Data Structure C, divide its accumulated assigned intensity by the accumulated assignable intensity to get its MS2SMILES score.
4. Return a list of SMILES strings and scores.

In particular aspects, while the precomputation step is performed one time for the entire database, it operates on one SMILES string at a time. Therefore, adding new strings to the database is readily accomplished as those strings become available from public or other sources.

4. A method for predicting the RI of a molecule based solely on its SMILES string or its molecular formula, based on a statistical analysis of compounds in the library.

The RI (retention index) of a compound is a property of an individual compound and is determined by the peculiarities of the particular LC process employed. RI depends on the three-dimensional structure of the compound (mainly what substructures are on the surface of the molecule interacting with the static and mobile phases of the LC setup). As such, RI generally cannot be predicted with high accuracy from the two-dimensional SMILES string. However, even a very rough prediction of RI is sufficient to eliminate many candidate structures for an unknown spectrum.

In alternate aspects, two methods can be implemented, one based solely on the molecular formula (“one-dimensional structure”) of the molecules, and another based on the SMILES string.

Method 1: RI Prediction by Linear Regression from Molecular Formula Only.
Precomputation: Use compounds from the library to create a simple linear regression model. Independent variables are the counts of atoms of the elements represented in the library compounds. The dependent variable is the RI. The coefficients of the linear model are made persistent.
Query: The coefficients of the linear model are applied to the counts of atoms of the query formula or SMILES, giving a predicted RI.

The second method uses an estimate of logP, the octanol-water partition coefficient, as an independent variable, logP is defined experimentally, but it can be estimated from a SMILES string using various software packages. The software package chosen for the exemplification herein is RdKit. As estimates vary among software packages, it is important to use the same software package for all estimates.

In addition, for an existing ion repository, SMARTS matching was used to count the number of primary amine groups (—NH₂, or *N in SMILES notation) in the SMILES strings. Adding this independent variable may improve accuracy.

In other aspects, other SMILES fragments may be later found that may correlate with RI and those SMILES fragments added as independent variables.

Method 2: RI Prediction by Linear Regression from SMILES.
Precomputation: Use compounds from the library to create a linear regression model. Independent variables are:
the counts of atoms of the elements represented in the library compounds.
logP, as estimated from SMILES by a software package.
number of primary amine groups, as detected by SMARTS searching or otherwise.
number of other functional groups, as detected by SMARTS searching or otherwise, to be determined by future analysis of libraries.
   The dependent variable is the RI. The coefficients of the linear model are made persistent.
   Query: The coefficients of the linear model are applied to the counts of atoms of the query formula or SMILES to derive a predicted RI.

Since these two methods implement statistical models built on relatively simple chemical properties of the compounds in the library, these methods may not necessarily be reliably extrapolated to formulas not statistically similar to the formulas in the implemented library (e.g., for formulas with low harmony).

### 5. GUI

A graphical report for the elucidation analysis herein is generated and displayed on a display for human evaluation, including color drawings of molecular structures.

Aspects of the present disclosure thus provide methods of analyzing and elucidating metabolomics data from a LC/tandem MS system, as disclosed herein. In addition to providing appropriate apparatuses and methods, aspects of the present disclosure also provide associated computer program products for performing the functions/operations/steps disclosed herein, in the form of, for example, a non-transitory computer-readable storage medium (i.e., memory device 140, FIG. 1) having particular computer-readable program code portions stored therein by the medium that, in response to execution by the processor device 130, cause the apparatus to at least perform the steps disclosed herein. In this regard, it will be understood that each block or step of the methodology or combinations of blocks/steps in the methodology can be implemented by appropriate computer program instructions executed by the processor device 130. These computer program instructions may be loaded onto a computer device or other programmable apparatus for executing the functions specified in the methodology or otherwise associated with the method(s) disclosed herein. These computer program instructions may also be stored in a computer-readable memory (i.e., memory device 140), so as to be accessible by a computer device or other programmable apparatus in a particular manner, such that the executable instructions stored in the computer-readable memory may produce or facilitate the operation of an article of manufacture capable of directing or otherwise executing the instructions which implement the functions specified in the methodology or otherwise associated with the method(s) disclosed herein. The computer program instructions may also be loaded onto a computer device or other programmable apparatus to cause a series of operational steps to be performed on the computer device or other programmable apparatus to produce a computer-implemented process such that the instructions executed by the computer device or other programmable apparatus provide or otherwise direct appropriate steps for implementing the functions/steps specified in the methodology or otherwise associated with the method(s) disclosed herein. It will also be understood that each step of the methodology, or combinations of steps in the methodology, can be implemented by special purpose hardware-based computer systems which perform the specified functions or steps, or combinations of special purpose hardware and computer instructions (software).

Many modifications and other embodiments of the inventions set forth herein will come to mind to one skilled in the art to which these disclosed embodiments pertain having the benefit of the teachings presented in the foregoing descriptions and the associated drawings. Therefore, it is to be understood that embodiments of the invention are not to be limited to the specific embodiments disclosed and that modifications and other embodiments are intended to be included within the scope of the invention. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the disclosure. In this regard, for example, different combinations of elements and/or functions than those explicitly described above are also contemplated within the scope of the disclosure. Although specific terms are employed herein, they are used in a generic and descriptive sense only and not for purposes of limitation.

It should be understood that although the terms first, second, etc. may be used herein to describe various steps or calculations, these steps or calculations should not be limited by these terms. These terms are only used to distinguish one operation or calculation from another. For example, a first calculation may be termed a second calculation, and, similarly, a second step may be termed a first step, without departing from the scope of this disclosure. As used herein, the term “and/or” and the “/” symbol includes any and all combinations of one or more of the associated listed items.

As used herein, the singular forms “a”, “an” and “the” are intended to include the plural forms as well, unless the context clearly indicates otherwise. It will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

1. A method of analyzing data for one or more samples, the data for each sample being obtained from a component separation and tandem mass spectrometer system including a mass spectrometer conducting a first mass spectrometry step or function (MS1) and a second mass spectrometry step or function (MS2), and structurally elucidating small molecule components of the one or more samples, said method comprising:
for each sample, determining a molecular mass of a fragment of a candidate compound;
determining possible molecular formulas having the molecular mass of the fragment;
aggregating MS2 spectra for each of a plurality of fragments of the candidate compound to form a candidate MS2 spectrum of the candidate compound;
determining possible molecular formulas and compound structures of an ion in the candidate MS2 spectrum, the ion comprising one or more fragments, that are consistent with the possible molecular formulas of the fragments;
determining known compounds having an MS2 spectrum similar to the candidate MS2 spectrum of the candidate compound;
determining known compounds having a compound structure plausibly corresponding to the MS2 spectrum of the candidate compound;
determining a probability of the MS2 spectrum of each fragment having one or more compound substructures;
determining a combination of known fragment spectra forming a compound spectrum statistically similar to the candidate MS2 spectrum of the candidate compound; and
associating the determined possible molecular formulas and compound structures, determined known compounds, determined compound substructures, and determined combination of known fragment spectra with the MS2 spectrum of the candidate compound and fragments thereof.

2. The method of claim 1, wherein determining possible molecular formulas having the molecular mass of the fragment, comprises determining arithmetically possible molecular formulas for the molecular mass of the fragment, with the arithmetically possible molecular formulas satisfying double-bond constraints, being statistically similar to molecular formulas of known metabolites, and satisfying isotopic constraints from MS1 analysis.

3. The method of claim 1, wherein determining possible molecular formulas and compound structures of an ion in the candidate MS2 spectrum consistent with the possible molecular formulas of the fragments thereof, comprises determining possible isomeric substructures corresponding to the possible molecular formulas and compound structures of the ion.

4. The method of claim 1, wherein determining known compounds having a compound structure plausibly corresponding to the MS2 spectrum of the candidate compound, comprises determining known compounds each having a SMILES string identifier plausibly corresponding to the MS2 spectrum of the candidate compound according to a measure of SMILES-to-spectrum similarity.

5. The method of claim 1, wherein determining known compounds having a compound structure plausibly corresponding to the MS2 spectrum of the candidate compound, comprises ranking plausible molecular formulas of the determined known compounds based on statistical similarity of the plausible molecular formulas to molecular formulas in a compound library.

6. The method of claim 1, wherein determining a probability of the MS2 spectrum of each fragment having one or more compound substructures, comprises predicting whether one or more compound substructures, each expressed as a SMILES string, is present in the fragment from the MS2 spectrum of each fragment.

7. An apparatus for analyzing data for one or more samples to structurally elucidate small molecule components thereof, the data for each sample being obtained from a component separation and tandem mass spectrometer system including a mass spectrometer for conducting a first mass spectrometry step or function (MS1) and a second mass spectrometry step or function (MS2), the apparatus comprising a processor and a memory storing executable instructions that, in response to execution by the processor, cause the apparatus to at least:
determine, for each sample, a molecular mass of a fragment of a candidate compound;
determine possible molecular formulas having the molecular mass of the fragment;
aggregate MS2 spectra for each of a plurality of fragments of the candidate compound to form a candidate MS2 spectrum of the candidate compound;
determine possible molecular formulas and compound structures of an ion in the candidate MS2 spectrum, the ion comprising one or more fragments, that are consistent with the possible molecular formulas of the fragments;
determine known compounds having an MS2 spectrum similar to the candidate MS2 spectrum of the candidate compound;
determine known compounds having a compound structure plausibly corresponding to the MS2 spectrum of the candidate compound;
determine a probability of the MS2 spectrum of each fragment having one or more compound substructures;
determine a combination of known fragment spectra forming a compound spectrum statistically similar to the candidate MS2 spectrum of the candidate compound; and
associate the determined possible molecular formulas and compound structures, determined known compounds, determined compound substructures, and determined combination of known fragment spectra with the MS2 spectrum of the candidate compound and fragments thereof.

8. A computer program product for analyzing data for one or more samples to structurally elucidate small molecule components thereof, the data for each sample being obtained from a component separation and tandem mass spectrometer system including a mass spectrometer for conducting a first mass spectrometry step or function (MS1) and a second mass spectrometry step or function (MS2), the computer program product comprising at least one non-transitory computer readable storage medium having computer-readable program code stored thereon, the computer-readable program code comprising program code for:
determining, for each sample, a molecular mass of a fragment of a candidate compound;
determining possible molecular formulas having the molecular mass of the fragment;
aggregating MS2 spectra for each of a plurality of fragments of the candidate compound to form a candidate MS2 spectrum of the candidate compound;
determining possible molecular formulas and compound structures of an ion in the candidate MS2 spectrum, the ion comprising one or more fragments, that are consistent with the possible molecular formulas of the fragments;
determining known compounds having an MS2 spectrum similar to the candidate MS2 spectrum of the candidate compound;
determining known compounds having a compound structure plausibly corresponding to the MS2 spectrum of the candidate compound;
determining a probability of the MS2 spectrum of each fragment having one or more compound substructures;
determining a combination of known fragment spectra forming a compound spectrum statistically similar to the candidate MS2 spectrum of the candidate compound; and
associating the determined possible molecular formulas and compound structures, determined known compounds, determined compound substructures, and determined combination of known fragment spectra with the MS2 spectrum of the candidate compound and fragments thereof.

9. The apparatus of claim 7, wherein the memory storing executable instructions that, in response to execution by the processor, cause the apparatus to determine possible molecular formulas having the molecular mass of the fragment, comprises the memory storing executable instructions that, in response to execution by the processor, cause the apparatus to determine arithmetically possible molecular formulas for the molecular mass of the fragment, with the arithmetically possible molecular formulas satisfying double-bond constraints, being statistically similar to molecular formulas of known metabolites, and satisfying isotopic constraints from MS1 analysis.

10. The apparatus of claim 7, wherein the memory storing executable instructions that, in response to execution by the processor, cause the apparatus to determine possible molecular formulas and compound structures of an ion in the candidate MS2 spectrum consistent with the possible molecular formulas of the fragments thereof, comprises the memory storing executable instructions that, in response to execution by the processor, cause the apparatus to determine possible isomeric substructures corresponding to the possible molecular formulas and compound structures of the ion.

11. The apparatus of claim 7, wherein the memory storing executable instructions that, in response to execution by the processor, cause the apparatus to determine known compounds having a compound structure plausibly corresponding to the MS2 spectrum of the candidate compound, comprises the memory storing executable instructions that, in response to execution by the processor, cause the apparatus to determine known compounds each having a SMILES string identifier plausibly corresponding to the MS2 spectrum of the candidate compound according to a measure of SMILES-to-spectrum similarity.

12. The apparatus of claim 7, wherein the memory storing executable instructions that, in response to execution by the processor, cause the apparatus to determine known compounds having a compound structure plausibly corresponding to the MS2 spectrum of the candidate compound, comprises the memory storing executable instructions that, in response to execution by the processor, cause the apparatus to rank plausible molecular formulas of the determined known compounds based on statistical similarity of the plausible molecular formulas to molecular formulas in a compound library.

13. The apparatus of claim 7, wherein the memory storing executable instructions that, in response to execution by the processor, cause the apparatus to determine a probability of the MS2 spectrum of each fragment having one or more compound substructures, comprises the memory storing executable instructions that, in response to execution by the processor, cause the apparatus to predict whether one or more compound substructures, each expressed as a SMILES string, is present in the fragment from the MS2 spectrum of each fragment.

14. The computer program product of claim 8, wherein the computer-readable program code comprising program code for determining possible molecular formulas having the molecular mass of the fragment, comprises program code for determining arithmetically possible molecular formulas for the molecular mass of the fragment, with the arithmetically possible molecular formulas satisfying double-bond constraints, being statistically similar to molecular formulas of known metabolites, and satisfying isotopic constraints from MS1 analysis.

15. The computer program product of claim 8, wherein the computer-readable program code comprising program code for determining possible molecular formulas and compound structures of an ion in the candidate MS2 spectrum consistent with the possible molecular formulas of the fragments thereof, comprises program code for determining possible isomeric substructures corresponding to the possible molecular formulas and compound structures of the ion.

16. The computer program product of claim 8, wherein the computer-readable program code comprising program code for determining known compounds having a compound structure plausibly corresponding to the MS2 spectrum of the candidate compound, comprises program code for determining known compounds each having a SMILES string identifier plausibly corresponding to the MS2 spectrum of the candidate compound according to a measure of SMILES-to-spectrum similarity.

17. The computer program product of claim 8, wherein the computer-readable program code comprising program code for determining known compounds having a compound structure plausibly corresponding to the MS2 spectrum of the candidate compound, comprises program code for ranking plausible molecular formulas of the determined known compounds based on statistical similarity of the plausible molecular formulas to molecular formulas in a compound library.

18. The computer program product of claim 8, wherein the computer-readable program code comprising program code for determining a probability of the MS2 spectrum of each fragment having one or more compound substructures, comprises program code for predicting whether one or more compound substructures, each expressed as a SMILES string, is present in the fragment from the MS2 spectrum of each fragment.
